(19) United States
(12) Patent Application Publication
(10) Pub. No.: US 2016/0122720 A1
(43) Pub. Date: May 5, 2016

(54) METHOD OF EFFICIENTLY ESTABLISHING INDUCED PLURIPOTENT STEM CELLS

(71) Applicant: KYOTO UNIVERSITY, Kyoto-shi, Kyoto (JP)

(72) Inventors: Shinya YAMANAKA, Kyoto-shi, Kyoto (JP); Kazutoshi TAKAHASHI, Kyoto-shi, Kyoto (JP); Koji TANABE, Kyoto-shi, Kyoto (JP); Mari OHNUKI, Kyoto-shi, Kyoto (US)

(21) Appl. No.: 14/897,801

(22) PCT Filed: Jun. 11, 2014
(86) PCT No.: PCT/JP2014/066080

§ 371 Date: Dec. 11, 2015

Related U.S. Application Data
(60) Provisional application No. 61/833,722, filed on Jun. 11, 2013.

(51) Int. Cl. C12N 5/074

(52) U.S. Cl. CPC C12N 5/0696; C12N 2501/603; C12N 2501/602; C12N 2501/604

(57) ABSTRACT

The invention provides a method of producing iPS cells, which comprises the steps of (i) introducing reprogramming factors into somatic cells; (ii) culturing the cells obtained in step (i) for more than 11 days and not more than 29 days; (iii) sorting TRA-1-60-positive cells from the cells obtained in step (ii); (iv) culturing the TRA-1-60-positive cells sorted in step (iii); (v) transferring a colony obtained in step (iv) to another culture vessel; and (vi) culturing the cells obtained in step (v), thereby obtaining iPS cells. The cells obtained in step (v) are preferably subcultured 10 times or more. The invention also provides a method of producing a population of differentiated cells that has a reduced rate of residual undifferentiated cells, which comprises inducing differentiation of the iPS cells obtained by the above-mentioned method.

This application is based on U.S. provisional patent application No. 61/833,722, the content of which is hereby incorporated by reference.

### TECHNICAL FIELD OF THE INVENTION

The present invention relates to a method of efficiently establishing induced pluripotent stem (hereinafter referred to as “iPS”) cells. The present invention also relates to a method of producing safe iPS cells that have a reduced risk of tumorigenesis when inducing differentiation.

### BACKGROUND OF THE INVENTION

Induced pluripotent stem cells (iPSCs) were first generated in 2006 by introducing a combination of four transcription factors, Oct3/4, Sox2, Klf4 and c-Myc (OSKM), into embryonic and adult mouse fibroblasts (1). Subsequently, human iPSCs were generated from fibroblasts using either the same factor combination (OSKM) or different (2), but overlapping, combinations of factors, such as OS plus LIN28 and NANOG (3). In addition to fibroblasts, iPSCs have been derived from various types of somatic cells, including hepatocytes, gastric epithelial cells (4), blood cells (5) and neural cells (6-8).

Although iPSCs can be reproducibly generated, only a small portion of somatic cells that receive the reprogramming factors become iPSCs. In our initial report (2), approximately 10 iPSC colonies emerged from 5×10⁵ fibroblasts that were re-plated seven days after the transduction of OSKM. This low efficiency (˜0.2%) raised the possibility that the origin of iPSCs is a rare population of stem or progenitor cells that co-exist in somatic cell culture. However, this possibility has been formally ruled out, because iPSCs can be generated from terminally differentiated T (9, 10) and B lymphocytes (5) that have undergone genetic recombination. However, the remaining important question is why only a small portion of transduced somatic cells can become iPSCs.

To answer this important question, it is critical to monitor the fate of cells transduced with OSKM during the course of reprogramming, which takes 20 to 30 days. To this end, it is essential to detect cells that are transduced and are subsequently reprogrammed. In mice, several studies have utilized a secondary iPSC induction system to uniformly introduce OSKM (6, 11) and specific markers, such as SSEA-1 (12, 13), to detect the cells being reprogrammed. These studies have focused on the molecular events that occur during reprogramming, and did not examine the cells that failed to become iPSCs. Furthermore, little is known about the molecular processes of human iPSC generation. In addition, the secondary iPSC induction system may be substantially different from the primary induction by the exogenous delivery of OSKM.

### REFERENCES

1. Takahashi, K. & Yamanaka, S. (2006) Cell 126, 663-676.
2. Takahashi, K. et al. (2007) Cell 131, 861-72.
3. Yu, J. et al. (2007) Science 318, 1917-20.
4. Aoi, T. et al. (2008) Science 321, 699-702.
5. Hanna, J. et al. (2008) Cell 133, 250-64.
6. Wernig, M. et al. (2008) Nat Biotechnol 26, 916-24.
7. Eminli, S. et al. (2008) Stem Cells 26, 2467-74.
8. Kim, J. et al. (2008) Nature 454, 646-50.
9. Loh, Y. et al. (2010) Cell Stem Cell 7, 15-9.
10. Seki, T. et al. (2010) Cell Stem Cell 7, 11-4.
11. Eminli, S. et al. (2009) Nat Genet 41, 968-76.
12. Buganim, Y. et al. (2012) Cell 150, 1209-22.
13. Polo, J. M. et al. (2012) Cell 151, 1617-32.

### SUMMARY OF THE INVENTION

In the present invention, we used a SOX2-transgene linked with enhanced green fluorescent protein (EGFP) with an internal ribosome entry site (IRES) to detect cells that had received the transgenes. We also monitored the cells for the expression of TRA-1-60, a glycoprotein that is expressed in human iPSCs and embryonic stem cells (ESCs), but not in somatic cells. TRA-1-60 is one of the best markers for human pluripotent stem cells (Chan, E. M. et al. (2009) Nat Biotechnol. 27, 1033-7; Andrews, P. W. et al. (1984) Hybridoma 3, 347-61.). By detecting and sorting the EGFP and/or TRA-1-60 (+) cells by flow cytometry, we tried to understand how nascent reprogrammed cells emerge and mature into iPSCs after retroviral transduction of OSKM into human dermal fibroblasts (HDFs) and why most of the transduced cells fail to become iPSCs.

As a result, we revealed that most HDFs initiated the reprogramming process upon receiving the OSKM transgenes. Within seven days, ˜20% of these transduced cells became positive for the TRA-1-60 antigen, however, only a small portion (˜1%) of these nascent reprogrammed cells resulted in colonies of induced pluripotent stem cells (iPSC) after re-plating. We found that many of the TRA-1-60 (+) cells turned back to be negative again during the subsequent culture. When TRA-1-60 (+) cells were sorted and re-plated on SNL feeder cells on day 7 or 11 after transduction, about a half of them reverted to a TRA-1-60 (−) state. In contrast, when they were sorted on day 15, the reversion rate became less than 10%. In addition, the efficiency of iPSC colony formation from TRA-1-60 (+) cells, which were sorted on day 7 or 11, remained low (˜1%). In contrast, the TRA-1-60 (+) cells sorted on day 15 or 20 showed a significantly increased efficiency of iPSC colony formation. These results indicate that nascent reprogrammed cells mature during this period (between day 11 and 15) and that iPS cell-establishing efficiency can be significantly improved by sorting and re-seeding TRA-1-60 (+) cells after this period.

Among the factors that have previously been reported to enhance direct reprogramming, LIN28, but not NANOG, Cyclin D1 or p53 shRNA, significantly inhibited the reversion of reprogramming. These data demonstrate that maturation, not initiation, is the rate limiting step during the direct reprogramming of human fibroblasts toward pluripotency, and that each pro-reprogramming factor has a different mode of action.

We also examined whether TRA-1-60 (+) cells sorted on or after day 21 retain undifferentiated cells after induction of differentiation. As a result, polyclonal TRA-1-60 (+) cells after 10 or more passages retained sufficiently reduced undifferentiated cells.

We have conducted further studies based on these findings, which resulted in the completion of the present invention.

That is, the present invention provides the following.

[1] A method of producing iPS cells, which comprises the following steps:

(i) a step for introducing reprogramming factors into somatic cells;

(ii) a step for culturing the cells obtained in step (i) for more than 11 days and not more than 29 days;

(iii) a step for sorting TRA-1-60-positive cells from the cells obtained in step (ii);

(iv) a step for culturing the TRA-1-60-positive cells sorted in step (iii);

(v) a step for transferring a colony obtained in step (iv) to another culture vessel; and

(vi) a step for culturing the cells obtained in step (v), thereby obtaining iPS cells.

[2] The method according to [1] above, wherein the reprogramming factors comprise:

(a) Oct3/4 or a nucleic acid encoding same;

(b) Sox2 or a nucleic acid encoding same; and

(c) Klf4 or a nucleic acid encoding same.

[3] The method according to [2] above, wherein the reprogramming factors further comprise (d) Lin28 or a nucleic acid encoding same.
[4] The method according to [1] above, wherein the iPS cells are human IFS cells.
[5] The method according to [1] above, wherein the culture period of step (ii) is 15 to 20 days.
[6] The method according to [1] above, wherein the cells obtained in step (vi) are subcultured 10 times or more.
[7] A method of producing a population of differentiated cells that has a reduced rate of residual undifferentiated cells, which comprises inducing differentiation of IFS cells obtained by the method according to [6] above.

The efficiency of establishing iPS cells can be improved by sorting TRA-1-60-positive cells after culturing cells into which reprogramming factors were introduced for more than 11 days after the introduction, because the TRA-1-60-positive cells are remarkably suppressed to revert to TRA-1-60-negative state. When the TRA-1-60-positive cells thus sorted are subcultured 10 times or more, the rate of residual undifferentiated cells are remarkably reduced when inducing differentiation, which enables the provision of safe cells for transplantation derived from IPS cells.

### BRIEF DESCRIPTION OF THE DRAWINGS

FIG. 1 shows the efficiency of iPSC induction.
A. The proportion of EGFP (+) cells 7 days after OSKM transduction into various HDF lines was analyzed by flow cytometry. The HDFs were derived from various ages (year; y, month; m) of Caucasian and Japanese males (M) and females (F). N=3. The error bars indicate the standard deviation.
B. The number of integrated retroviral transgenes per TRA-1-60 (+), EGFP (+)/TRA-1-60 (−) and EGFP (−)/TRA-1-60 (−) cell on day 7, 11 and 15 was analyzed by a quantitative genomic PCR analysis. Parental non-transduced HDFs were used as a negative control. N=3. The error bars indicate the standard deviation.
C. The number of iPSC colonies derived from 2.5×10⁵ HDFs transduced with OSKM on day 24. The HDFs were derived from various ages (year; y, month; m) of Caucasian and Japanese males (M) and females (F). N=3. The error bars indicate the standard deviation.

FIG. 2 shows characterization of the TRA-1-60 (+) cells.
A. The proportion of TRA-1-60 (+) cells in the population of EGFP (+) cells on day 7. The HDFs were derived from various ages (year; y, month; m) of Caucasian and Japanese males (M) and females (F). N=3. The error bars indicate the standard deviation.
B. The number of iPSC colonies derived from 2.5×10⁵ TRA-1-60 (+), TRA-1-60 (−) or EGFP (−) cells transduced with OSKM on day 11, 21 days after sorting (on day 32). N=3. The error bars indicate the standard deviation.
C. A heat map of the HDF-G or ES-G expression in HDFs, ESCs, EGFP (+)/TRA-1-60 (−) and EGFP (−)/TRA-1-60 (−) cells on day 7, 11 and 15 and in TRA-1-60 (+) cells on day 7, 11, 15, 20 and 28. N=3. The error bars indicate the standard deviation.
D. The relative expression level of pluripotency genes in TRA-1-60 (+) cells transduced with OSKM on day 7, 11 and 15 post-transduction was analyzed by qRT-PCR. All values are normalized to the expression in ESCs. N=3. The error bars indicate the standard deviation.
E. The left Venn diagram indicates the overlap of the 10-fold increased ES-Gs between TRA-1-60 (+) and EGFP (+)/TRA-1-60 (−) cells compared to HDFs. The right Venn diagram shows the overlap of the 10-fold decreased HDF-Gs between the TRA-1-60 (+) and EGFP (+)/TRA-1-60 (−) cells compared to HDFs.
F. The PCA on the gene expression levels of ES-Gs and HDF-Gs in TRA-1-60 (+), EGFP (+)/TRA-1-60 (−) and EGFP (−)/TRA-1-60 (−) cells. N=3.

FIG. 3 shows the results of the single cell expression analysis during reprogramming.
A. A heat map of the gene expression in each single cell was determined using the Biomark system. TRA-1-60 (−)/EGFP (+) or (−) cells were sorted on day 7, 11 and 15 post-transduction. TRA-1-60 (+) cells were sorted on day 7, 11, 15, 20 and 28 post-transduction. The heat map shows the Ct values in a single cell qRT-PCR from cycles 12 to 26. The black marks indicate undetectably low expression, which was defined as when the Ct values were higher than 26.
B. Violin plots of the Ct value of the gene expression in same single cells shown in FIG. 3A. The white dots indicate the median values. Ct 30 indicates undetectable expression, which was indicated by Ct values higher than 26.
C. The PCA of the gene expression levels in each of the individual cells shown in FIG. 3A.
D. The variability of gene expression in the TRA-1-60 (+) cells shown in FIG. 3A was determined using the JSD. The error bars indicate the 95% confidence intervals.

FIG. 4 shows reversion during iPSC induction.
A. The generation efficiency of iPSC colonies derived from TRA-1-60 (+) cells on day 7, 11, 15 and 20. TRA-1-60 (+) cells were seeded on feeder cells. The numbers of iPSC colonies were counted 21 days after seeding. N=3. The error bars indicate the standard deviation.
B. The proportion of reverted TRA-1-60 (−) cells from TRA-1-60 (+) cells. TRA-1-60 (+) cells were sorted and seeded on feeder cells on different days (day 7, 11 and 15). The proportions of TRA-1-60 (−) cells in the TRA-1-85 (+) cell population were analyzed 4 days after seeding. The human specific marker, TRA-1-85, was used to distinguish human cells from the mouse feeder cells. N=3. The error bars indicate the standard deviation.
C. The PCA of the expression of ES-Gs and HDF-Gs during reprogramming and reversion from the microarray data. The black circles indicate the gene expression patterns of reprogramming cells from HDFs to iPSCs/ESCs. Day 3: EGFP (+) cells. Day 7, 11, 15 and 28: TRA-1-60 (+) cells on each day post-transduction. The colored circles indicate the expression patterns of the reverted TRA-1-60 (−) cells on day (Green) and 20 (Magenta) compared to the TRA-1-60 (+) cells on day 11. The colored squares indicate non-reverted TRA-1-60 (+) cells on each day.

FIG. 5 shows the effect of pro-reprogramming factors on reprogramming.
A. The number of iPSC colonies on day 24 formed from HDFs treated with OSKM plus different pro-reprogramming factors. The P values were calculated using T-tests comparing the different groups to cells treated with OSKM alone (Mock); N=3 Asterisks indicate p<0.05.
B. The relative proportion of BrdU (+) cells in TRA-1-60 (−) cells on day 7. All values are normalized to those of cells transduced with OSKM alone (Mock). N=3. The error bars indicate the standard deviation.
C. The relative proportion of TRA-1-60 (+) cells on day 7 post-transduction. All values are normalized to those of cells transduced with OSKM alone (Mock). N=3. The error bars indicate the standard deviation.
D. The relative proportion of BrdU incorporation into TRA-1-60 (+) cells on day 7. All values are normalized to those of cells transduced with OSKM alone (Mock). N=3. The error bars indicate the standard deviation.
E. The proportion of apoptotic cells in TRA-1-60 (+) cells on day 11. The TRA-1-60 (+) cells were immunostained with Annexin V. N=3. The error bars indicate the standard deviation.
F. The effects of each pro-reprogramming factor on the reversion of TRA-1-60 positive to negative state. The proportion of TRA-1-60 (−) cells in the total population of TRA-1-85 (+) cells 4 days after seeding the TRA1-60 (+) cells sorted on day 11 on feeder cells. N=3. The error bars indicate the standard deviation.

FIG. 6 shows a model of the reprogramming process. HDFs (black dots) were introduced OKM plus SOX2-IRES-EGFP using a retroviral system. About 6 to 20% of the cells were successfully introduced with the reprogramming factors. Reprogramming was initiated in the majority of the EGFP (+) cells (green dots). Non-transduced cells (black dots) did not initiate reprogramming. About 12 to 24% of the transduced cells became TRA-1-60 (+) (magenta dots). The reprogramming progressed in the TRA-1-60 (+) cells, but not in the EGFP (+)/TRA-1-60 (−) cells. During the maturation step, at least 50% of the TRA-1-60 (+) cells underwent reversion to become TRA-1-60 (−) after four days. Reversion was inhibited by LIN28. Less than 0.2% of the total number of HDFs formed iPSC colonies because of the bottleneck of reprogramming occurring during the maturation steps. Transduced cells underwent apoptosis during both the initiation and maturation steps.

### DETAILED DESCRIPTION OF THE INVENTION

Induced pluripotent stem (iPS) cell is an artificial stem cell derived from a somatic cell, which can be produced by introducing a specific reprogramming factor in the form of a DNA or protein into a somatic cell, and show almost equivalent property (e.g., pluripotent differentiation and proliferation potency based on self-renewal) as ES cells (K. Takahashi and S. Yamanaka (2006) Cell, 126:663-676; K. Takahashi et al. (2007), Cell, 131:861-872; J. Yu et al. (2007), Science, 318:1917-1920; Nakagawa, M. et al., Nat. Biotechnol. 26:101-106 (2008); WO2007/069666).

The term “somatic cell” used in the present specification means any animal cell (preferably, cells of mammals inclusive of human) excluding germ line cells and totipotent cells such as ovum, oocyte, ES cells and the like. Somatic cell unlimitatively encompasses any of somatic cells of fetuses, somatic cells of neonates, and mature healthy or pathogenic somatic cells, and any of primary cultured cells, passage cells, and established lines of cells. Specific examples of the somatic cell include (1) tissue stem cells (somatic stem cells) such as neural stem cell, hematopoietic stem cell, mesenchymal stem cell, dental pulp stem cell and the like, (2) tissue progenitor cell, (3) differentiated cells such as lymphocyte, epithelial cell, endothelial cell, myocyte, fibroblast (skin cells etc.), hair cell, hepatocyte, gastric mucosal cell, enterocyte, splenocyte, pancreatic cell (pancreatic exocrine cell etc.), brain cell, lung cell, renal cell and adipocyte and the like, and the like.

The choice of mammal individual as a source of somatic cells is not particularly limited; however, when the iPS cells are to be used for the treatment of diseases in humans, it is preferable, from the viewpoint of prevention of graft rejection and/or GvHD, that somatic cells are patient's own cells or collected from another person having the same or substantially the same HLA type as that of the patient. “Substantially the same HLA type” as used herein means that the HLA type of donor matches with that of patient to the extent that the transplanted cells, which have been obtained by inducing differentiation of iPS cells derived from the donor's somatic cells, can be engrafted when they are transplanted to the patient with use of immunosuppressor and the like. For example, it includes an HLA type wherein major HLAs (the three major loci of HLA-A, HLA-B and HLA-DR or four loci further including HLA-Cw) are identical (hereinafter the same meaning shall apply) and the like.

The reprogramming factor may be constituted with a gene specifically expressed by ES cell, a gene product or non-coding RNA thereof, a gene playing an important role for the maintenance of undifferentiation of ES cell, a gene product or non-coding RNA thereof, or a low molecular weight compound. Examples of the gene contained in the reprogramming factor include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tc11, beta-catenin, Lin28b, Sall1, Sall4, Esrrb, Nr5a2, Tbx3, Glis1 and the like. These reprogramming factors may be used alone or in combination. Examples of the combination of the reprogramming factors include combinations described in WO2007/069666, WO2008/118820, WO2009/007852, WO2009/032194, WO2009/058413, WO2009/057831, WO2009/075119, WO2009/079007, WO2009/091659, WO2009/101084, WO2009/101407, WO2009/102983, WO2009/114949, WO2009/117439, WO2009/126250, WO2009/126251, WO2009/126655, WO2009/157593, WO2010/009015, WO2010/033906, WO2010/033920, WO2010/042800, WO2010/050626, WO2010/056831, WO2010/068955, WO2010/098419, WO2010/102267, WO2010/111409, WO2010/111422, WO2010/115050, WO2010/124290, WO2010/147395, WO2010/147612, Huangfu D, et al. (2008), Nat. Biotechnol., 26: 795-797, Shi Y, et al. (2008), Cell Stem Cell, 2: 525-528, Eminli S, et al. (2008), Stem Cells.

26:2467-2474, Huangfu D, et al. (2008), Nat Biotechnol. 26:1269-1275, Shi Y, et al. (2008), Cell Stem Cell, 3, 568-574, Zhao Y, et al. (2008), Cell Stem Cell, 3:475-479, Marson A, (2008), Cell Stem Cell, 3, 132-135, Feng B, et al. (2009), Nat Cell Biol. 11:197-203, R. L. Judson et al., (2009), Nat. Biotech., 27:459-461, Lyssiotis C A, et al. (2009), Proc Natl Acad Sci USA. 106:8912-8917, Kim J B, et al. (2009), Nature. 461:649-643, Ichida J K, et al. (2009), Cell Stem Cell. 5:491-503, Heng J C, et al. (2010), Cell Stem Cell. 6:167-74, Han J, et al. (2010), Nature. 463:1096-100, Mali P, et al. (2010), Stem Cells. 28:713-720, and Maekawa M, et al. (2011), Nature. 474:225-9.

In a preferable embodiment, Oct3/4, Sox2 and Klf4 (OSK) can be used for reprogramming substances. More preferably, in addition to the three factors, a Myc family member (M) selected from L-Myc, N-Myc and c-Myc (including T58A mutant) can be used. Furthermore, as shown in Examples below, since Lin28 promotes the formation of TRA-1-60-positive cells and inhibits their conversion back into TRA1-60-negative cells, it is also preferable to use Lin28 as a reprogramming substance in addition to the three (OSK) or four (OSKM) factors.

Examples of the above-mentioned reprogramming factor include, but are not limited to, factors used for enhancing the establishment efficiency, such as histone deacetylase (HDAC) inhibitors [e.g., low-molecular inhibitors such as valproic acid (VPA), trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29 mer shRNA Constructs against HDAC1 (OriGene) and the like), and the like], MEK inhibitor (e.g., PD184352, PD98059, U0126, SL327 and PD0325901), Glycogen synthase kinase-3 inhibitor (e.g., Bio and CHIR99021), DNA methyl transferase inhibitors (e.g., 5-azacytidine), histone methyl transferase inhibitors [for example, low-molecular inhibitors such as BIX-01294, and nucleic acid-based expression inhibitors such as siRNAs and shRNAs against Suv39hl, Suv39h2, SetDB1 and G9a], L-channel calcium agonist (for example, Bayk8644), butyric acid, TGFβ inhibitor or ALK5 inhibitor (e.g., LY364947, SB431542, 616453 andA-83-01), p53 inhibitor (for example, siRNA and shRNA against p53), ARID3A inhibitor (e.g., siRNA and shRNA against ARID3A), miRNA such as miR-291-3p, miR-294, miR-295, mir-302 and the like, Wnt Signaling (for example, soluble Wnt3a), neuropeptide Y, prostaglandins (e.g., prostaglandin E2 and prostaglandin J2), hTERT, SV40LT, UTF1, IRX6, GLIS1, PITX2, DMRTB1 and the like. In the present specification, these factors used for enhancing the establishment efficiency are not particularly distinguished from the reprogramming factor.

When the reprogramming factor is in the form of a protein, it may be introduced into a somatic cell by a method, for example, lipofection, fusion with cell penetrating peptide (e.g., TAT derived from HIV and polyarginine), microinjection and the like.

When the reprogramming factor is in the form of a DNA, it may be introduced into a somatic cell by the method using, for example, vector of virus, plasmid, artificial chromosome and the like, lipofection, liposome, microinjection and the like. Examples of the virus vector include retrovirus vector, lentivirus vector (Cell, 126, pp. 663-676, 2006; Cell, 131, pp. 861-872, 2007; Science, 318, pp. 1917-1920, 2007), adenovirus vector (Science, 322, 945-949, 2008), adeno-associated virus vector, Sendai virus vector (vector of Hemagglutinating Virus of Japan) (WO 2010/008054) and the like. Examples of the artificial chromosome vector include human artificial chromosome (HAC), yeast artificial chromosome (YAC), bacterial artificial chromosome (BAC, PAC) and the like. As the plasmid, plasmids for mammalian cells can be used (Science, 322:949-953, 2008). The vector can contain regulatory sequences of promoter, enhancer, ribosome binding sequence, terminator, polyadenylation site and the like so that a nuclear reprogramming substance can be expressed and further, where necessary, a selection marker sequence of a drug resistance gene (for example, kanamycin resistance gene, ampicillin resistance gene, puromycin resistance gene and the like), thymidine kinase gene, diphtheria toxin gene and the like, a reporter gene sequence of green fluorescent protein (GFP), β glucuronidase (GUS), FLAG and the like, and the like. Moreover, the above-mentioned vector may have a LoxP sequence before and after thereof to simultaneously cut out a gene encoding a reprogramming factor or a gene encoding a reprogramming factor bound to the promoter, after introduction into a somatic cell.

When in the form of RNA, for example, it may be introduced into a somatic cell by means of lipofection, microinjection and the like, and RNA incorporating 5-methylcytidine and pseudouridine (TriLink Biotechnologies) may be used to suppress degradation (Warren L, (2010) Cell Stem Cell. 7:618-630).

Examples of the culture medium for inducing iPS cells include 10-15% FBS-containing DMEM, DMEM/F12 or DME culture medium (these culture media can further contain LIF, penicillin/streptomycin, puromycin, L-glutamine, nonessential amino acids, β-mercaptoethanol and the like as appropriate) or a commercially available culture medium [for example, culture medium for mouse ES cell culture (TX-WES culture medium, Thromb-X), culture medium for primate ES cell (culture medium for primate ES/iPS cell, Reprocell), serum-free medium (mTeSR, Stemcell Technologies)] and the like.

The present invention is, in part, based on the finding that until 11 days after introduction of reprogramming factors, approximately half or more of the reprogramming cells that have become TRA-1-60-positive revert to TRA-1-60-negative during the subsequent culture and do not ultimately become iPS cells, on the other hand, when culturing the cells for more than 11 days followed by sorting of TRA-1-60-positive cells and the subsequent culture, about 90% or more of the cells maintain TRA-1-60-positive state and are established as iPS cells.

Accordingly, the inventive method comprises:
(i) a step for introducing reprogramming factors into somatic cells;
(ii) a step for culturing the cells obtained in step (i) for more than 11 days; and
(iii) a step for sorting TRA-1-60-positive cells from the cells obtained in step (ii).

Examples of the culture method of step (i) above include contacting a somatic cell with a reprogramming factor on 10% FBS-containing DMEM or DMEM/F12 culture medium at 37° C. in the presence of 5% CO₂. A culture method using a serum-free medium can also be recited as an example (Sun N, et al. (2009), Proc Natl Acad Sci USA. 106:15720-15725). Furthermore, to enhance establishment efficiency, an iPS cell may be established under hypoxic conditions (oxygen concentration of not less than 0.1% and not more than 15%) (Yoshida Y, et al. (2009), Cell Stem Cell. 5:237-241 or WO2010/013845).

The culture medium is exchanged with a fresh culture medium once a day during the above-mentioned cultures, from day 2 from the start of the culture. While the cell number of the somatic cells used for nuclear reprogramming is not limited, it is about 5×10³-about 5×10⁶ cells per 100 cm² culture dish.

After the introduction, the cells can be cultured in the same medium as used in step (i) for more than 11 days. The culture period of step (ii) is not limited as long as it is more than 11 days, for example, 12 days or more, 13 days or more, 14 days or more, or 15 days or more, preferably 15 days or more. The upper limit of the culture period is not also limited, however, since only completely reprogrammed iPS cell colonies remain when culturing for 30 days or more, it is substantially meaningless to sort TRA-1-60-positive cells. Therefore, the culture period is 29 days or less, preferably 25 days or less, more preferably 20 days or less.

The sorting of TRA-1-60-positive cells can be, for example, carried out by flowcytometry using a commercially available anti-TRA-1-60 antibody.

The sorted TRA-1-60-cells can be (iv) reseeded on feeder cells (e.g., mitomycin C-treated STO cells, SNL cells etc.) or a dish coated by an extracellular substrate and cultured in a bFGF-containing culture medium for primate ES cell. The cells can also be cultured on feeder cells at 37° C. in the presence of 5% CO₂ in a 10% FBS-containing DMEM culture medium (which can further contain LIF, penicillin/streptomycin, puromycin, L-glutamine, nonessential amino acids, β-mercaptoethanol and the like as appropriate).

Alternatively, a method using somatic cells themselves to be reprogrammed, or an extracellular substrate (e.g., Laminin-5 (WO2009/123349) and Matrigel (BD)), instead of the feeder cells (Takahashi K, et al. (2009), PLoS One. 4:e8067 or WO2010/137746), can be mentioned.

The present invention further comprises:

(v) a step for picking up a colony obtained in step (iv) and transferring it to another culture vessel; and

(vi) a step for further culturing the cells obtained in step (v), thereby obtaining iPS cells.

In step (v), single colonies (clones) can be separately picked up, and each colony (clone) can be subcultured in a separate culture vessel. Alternatively, a plurality of colonies can be picked up and transferred together in another culture vessel and cultured in bulk.

When colonies are picked up polyclonally and subcultured in bunk 10 times or more, the rate of residual undifferentiated cells can be remarkably reduced when inducing differentiation into somatic cells. Therefore, safe iPS cells can be provided without selecting a clone having a reduced risk of tumorigenesis. In the present invention, “subculture” means dissociating iPS cells from a culture vessel and transferring all or about ½, ⅓ or ¼ of the cells to another culture vessel.

The present invention is hereinafter described in further detail by means of the following examples, to which, however, the invention is never limited.

### Examples

### Materials and Methods

### The Policy on the Statistical Analyses

All of the quantitative experiments were performed in at least as biological triplicates. In all figures, the asterisks indicate P-values <0.05 as determined by a paired t-test. The error bars indicate the standard deviations. The definition of HDF-G and ES-G was a fold-change (FC) >100, P<0.05, based on a comparison of 10 independent HDF lines and 10 independent ESC lines (Table 1).

TABLE 1
The list of cell lines used

| Cell | Cell | | | | | |
|---|---|---|---|---|---|---|
| types | name | Race | Sex | Age | | Provider |
| HDF | TIG107 | Japanese | Female | 81 | years | Japanese Collection |
| | TIG113 | Japanese | Female | 21 | years | of Research Bio- |
| | TIG118 | Japanese | Female | 1 | years | resources (Japan) |
| | TIG119 | Japanese | Male | 6 | years | |
| | TIG120 | Japanese | Female | 6 | years | |
| | TIG121 | Japanese | Male | 8 | months | |
| | 1388 | Caucasian | Female | 36 | years | Cell applications |
| | 1454 | Caucasian | Male | 0 | years | Inc. (USA) |
| | 1503 | Caucasian | Female | 73 | years | |
| | 1616 | Japanese | Female | 68 | years | |

| | | | | | |
|---|---|---|---|---|---|
| hES | KhES1 | Japanese | Female | Embryo | Kyoto University |
| | KhES3 | Japanese | Male | Embryo | |
| | H1 | | Male | Embryo | WiCELL (USA) |
| | H9 | | Female | Embryo | |
| | ES04 | | Male | Embryo | |
| | ES06 | | Female | Embryo | |
| | SA002 | | Female | Embryo | |

### Cell Culture

HDF lines were purchased from the Japanese Collection of Research Bioresources and Cell Applications Inc. The HDFs were maintained in Dulbecco's modified eagle medium (DMEM, Nacalai tesque) containing 10% fetal bovine serum (FBS, Thermo) and 0.5% penicillin and streptomycin (Pen/Strep, Invitrogen). PLAT-E cells were cultured 10% FBS medium with 1 μg/mi puromycin and 10 μg/ml blasticidin S. The ESC lines were obtained from Kyoto University and WiCELL, and were maintained in Human ESC medium (ReproCELL) supplemented with 4 ng/ml basic fibroblast growth factor (Wako) on mitomycin C-treated SNL feeder cells. All of the cell lines used are listed in Table 1.

### Plasmid Construction

The open reading frames (ORF) of the genes used in this study were amplified by PCR, subcloned into the pENTR-D-TOPO vector (Invitrogen) and verified by sequencing. After that, the ORFs were transferred into the pMXs-gw retroviral vector by using a Gateway LR reaction (Invitrogen) according to the manufacturer's protocol. The knockdown vector for TP53 was obtained from Addgene (#10672).

iPSC Colony Formation

Reprogramming was carried out as described in Takahashi & Yamanaka (Cell 126, 663-676 (2006)). To generate retroviruses, we introduced retroviral vectors encoding each factor into PLAT-E cells by using the FuGENE 6 transfection reagent as per the manufacturer's recommendations. On the following day, we changed the medium to fresh 10% FBS-containing medium and incubated the cells for about 24 h. The medium, including the virus, was then collected and filtered using a 0.45 μm pore size cellulose acetate filter (Whatman). Then, we mixed appropriate combinations of viruses and used them to expose HDFs expressing the mouse Slc7a1 gene overnight with 4 μg/ml polybrene (Nacalai tesque). We designated this point as day 0. Transduced cells were cultured with 10% FBS-containing medium for 7 days. We harvested the cells on day 7 post-transduction, and re-plated 2.5×10⁵ cells onto mitomycin C-inactivated SNL feeder cells. The next day, the medium was replaced with human ESC medium. The medium was changed every other day. We counted the number of iPSC colonies on day 24 post-transduction.

### Analysis of TRA-1-60 (+), EGFP (+)/TRA-1-60 (−) and EGFP (−)/TRA-1-60 (−) Cells

The cells transduced with OKM plus SOX2-IRES-EGFP were cultured with 10% FBS-containing medium for 8 days. The culture medium was then replaced with human ESC medium. On day 7, 11 and post-transduction, the transduced cells were harvested using 0.25% trypsin/1 mM EDTA and were filtered using a 70 μm pore size cell strainer (BD biosciences). The cells were then treated with an anti-TRA-1-60 MicroBead Kit (Miltenyi biotec) and sorted by their TRA-1-60 (+) cells by an auto MACS pro device (Miltenyi biotec). EGFP (+)/TRA-1-60 (−) and EGFP (−)/TRA-1-60 (−) cells were sorted by a FACS Aria II instrument (BD biosciences) from the TRA-1-60 (−) fraction after MACS. To collect the TRA-1-60 (+) cells on day 20 and 28 post-transduction, we reseeded the MACS-sorted 5×10⁵ TRA-1-60 (+) cells on mitomycin C-inactivated SNL feeders in 10 cm culture dishes on day 11 post-transduction. Then, the cells were cultured with Y-27632 (10 μM) in human ESC medium for 2 days. The media was then replaced with fresh human ESC medium every 2 days. On days 20 and 28, the TRA-1-60 (+) cells were sorted using the MACS protocol as described above.

### Sorting and Culturing Single TRA-1-60 (+) Cells

The TRA-1-60 (+) cells were sorted using the MACS protocol as described above. The sorted TRA-1-60 (+) cells were stained with DAPI (Life Technologies Corporation) for 30 min to detect the dead cells. Each of the TRA-1-60 (+)/DAPI (−) cells was directly sorted into a well of a 96 well plate on mitomycin C-inactivated SNL feeders using the FACS Aria II instrument. The cells were cultured in human ESC medium with Y-27632 (10 μM). Two days after sorting, we added fresh human ESC medium with Y-27632. We started to replace the medium every 2 days from 4 days after sorting. We counted the number of well in which there were iPSC colonies on day 32 post-transduction.

### Flow Cytometry and Fluorescence Activated Cell Sorting (FACS)

Transduced cells were harvested with 0.25% trypsin/1 mM EDTA on each day after transduction for the analysis. At least 1×10⁵ cells were stained with the following antibodies in FACS buffer (2% FBS, 0.36% Glucose in PBS) for 30 min at room temperature. The following antibodies were used for the analysis. Alexa 647-conjugated TRA-1-60 (1:20, 560122, BD biosciences), Alexa-488-conjugated TRA-1-60 (1:20, 560173, BD biosciences), phycoerythrin-conjugated TRA-1-85 (1:10, FAB3195P, R&D Systems),

### Analysis of Reversion

We sorted the TRA-1-60 (+) cells using the MACS protocol as described above. The TRA-1-60 (+) cells were cultured with human ESC medium with Y-27632 (10 μM) on mitomycin C-inactivated SNL feeders for 2 days. TRA-1-60 (+) cells were thereafter cultured for either another 2 days or 7 days (until day 15 or post-transduction). The media were replaced with fresh human ESC medium every 2 days. To detect the reversion to a TRA-1-60 (−) state, the transduced cells were stained with TRA-1-85 and TRA-1-60 antibodies as previously described in the FACS protocol. The proportion of TRA-1-60 (−) cells in the TRA-1-85 (+) population were calculated to detect the reversion. Reverted TRA-1-60 (−)/TRA-1-85 (+) cells were sorted using the FACS Aria II instrument prior to the microarray.

### Single Cell Gene Expression Analysis

We first made 0.2×Taqman probe mix (19 Taqman probes (Application Binary Interface); 1 μl×19, DNA suspension buffer (Tecnova); 4 μl, Water; 77 μl). The Taqman probes used in the study are listed up in Table 2.

TABLE 2
The list of Taqman probes for single cell PCR

| Probe Name | Probe ID |
|---|---|
| ACTB | Hs00357333_g1 |
| NANOG | Hs02387400_g1 |
| LITD1 | Hs00219458_m1 |
| GDF3 | Hs00220998_m1 |
| GAL | Hs00544355_m1 |
| SALL4 | Hs00360675_m1 |
| APOE | Hs00171168_m1 |
| DAPP4 | Hs01060238_g1 |
| SOX2 | Hs01053049_s1 |
| LIN28A | Hs00702808_s1 |
| GABRB3 | Hs00326767_s1 |
| DNMT3B | Hs01003405_m1 |
| CDH1 | Hs01013958_m1 |
| EPCAM | Hs00901885_m1 |
| MMP1 | Hs00899658_m1 |
| DCN | Hs00370385_m1 |
| LUM | Hs00158940_m1 |
| ANPEP | Hs00174265_m1 |

Single cells were directly sorted in 9 μl of master mix (Cells Direct 2×Reaction mix (Life Technologies Corporation), 5 μl of 0.2×Taqman probe mix, 2.5 μl of Super script III RT/Platinum Taq mix (Life Technologies Corporation) and 0.2 μl of DNA suspension buffer (Tecnova); 1.3 μl) using the FACS Aria II instrument. The reaction mixture was incubated in a thermal cycler for single cell lysis and reverse transcription at 50° C. for 15 min and for inactivation of reverse transcriptase at 95° C. for 2 min. cDNAs were amplified specifically in TaqMan assays at 95° C. for 15 sec and 60° C. for 4 min for 22 cycles. Single cell qPCR was performed with TaqMan assays, and the amplified cDNAs, which were diluted by 5-fold in 48.48 Dynamic Arrays on a BioMark System (Fluidigm). The Ct values were calculated by the software program provided by the manufacturer (Fluidigm Real-Time PCR Analysis). If Ct values were higher than 26, the expression was filtered out as undetectable/low expression. All TaqMan assays were checked to confirm that they could quantitate the gene expression at the single cell level.

### Microarrays

The total RNA was labeled with Cyanine 3. Samples were hybridized with the Whole Human Genome Microarray SurePrint G3 Human GE 8×60K (G4112F, Agilent technologies). Each sample was hybridized once using the one color protocol. The arrays were scanned with a G2565BA Microarray Scanner System (Agilent technologies). All of the microarray results were analyzed using the GeneSpring v 11 software program (Agilent technologies). Samples were normalized by 75 percentile shift. Entities were filtered by percentile. If at least one of samples had values within 100 to the 20 percentile, the entities were passed through the filter. Moreover, entities were filtered based on flag values. If the entities had a Present or Marginal value in at least one of the samples, the entities passed the filter.

BrdU incorporation

The medium was changed to fresh media one day before the analyses. On the next day, the cells were incubated with 10 μM BrdU for 30 min at 37° C. Then, the cells were harvested using 0.25% Trypsin/1 mM EDTA and were incubated with the anti-TRA1-60 antibody for 30 min at room temperature as previously described in the FACS protocol. The BrdU incorporation was detected with a BrdU Flow Kit (BD Pharmingen).

### Apoptosis

The cells transduced with OSKM were harvested on day 11 using 0.25% Trypsin/1 mM EDTA. They were then immediately stained with ApoAlert (Clontech), and staining was detected using the FACS Aria II instrument.

### JSD

The calculation was performed according to Buganim et al. (Cell 150, 1209-22 (2012)).

### SFEBq Method

SFEBq method was performed as follows. The obtained cells were incubated using Accumax™ for 5 min at 37° C. to dissociate, washed and the number of the cells was counted. The cells were suspended in the aforementioned differentiation medium and seeded onto a low attachment 96-well plate (Lipidure-coat plate: NOF Corporation) at 9000 cells/well. The cells were cultured in GMEM (Invitrogen) containing 10 μM Y-27632 (WAKO), 0.1 μM LDN193189 (STEMGENT), 0.5 μM A83-01 (WAKO), 8% KSR (Invitrogen), 1 mM Sodium pyruvate (Invitrogen), 0.1 mM MEM non essential amino acid (Invitrogen) and 0.1 mM 2-Mercaptoethanol (WAKO) for 14 days. Y27632 was added for the initial culture. The medium was not exchanged until day 7, thereafter exchanged every 3 days. After induction into nerve precursor cells, the cells were dispersed to single cells, and the number of TRA-1-60 (+) cells was determined using a flowcytometer.

### Results

We introduced pMXs retroviral vectors for OCT3/4, KLF4 and c-MYC, together with another pMXs vector expressing SOX2-IRES-EGFP, into 10 HDF lines, which were derived from donors of various ages (0-81 years old), including four Caucasian and six Japanese donors. On day seven after transduction, ˜20% (5.9 ˜24.5%) of the HDFs became EGFP (+) (FIG. 1A). We sorted the EGFP (+) cells and estimated the copy numbers of the transduced retroviruses by quantitative polymerase chain reaction (PCR). On day 11 or 15 after transduction, we detected approximately five copies/cell of retroviruses for each transgene (OCT3/4, SOX2, KLF4, or c-MYC) in the EGFP (+) cells (FIG. 1B). In contrast, in EGFP negative (−) cells, we detected only a few copies/cell of retroviruses. On day 7, we detected approximately twice as many copy numbers of retroviruses in both the EGFP (+) and (−) cells. The reason for the seemingly higher copy numbers on day 7 is unclear. Nevertheless, the results confirmed that EGFP (+) cells represented HDFs that had received higher numbers of retroviral OSKM, whereas EGFP (−) HDFs had integrated significantly fewer copies of retroviral transgenes.

On day seven, we re-plated 2.5×10⁵ cells onto SNL feeder cells and replaced the fibroblast medium with that for pluripotent stem cells. On day 24, we observed 9-583 iPSC colonies (FIG. 1C). Therefore, the putative efficiency of iPSC generation was low, ranging from 0.0036 to 0.23% from re-plated HDFs (FIG. 10), similar to the previously reported results (2).

In contrast to the low efficiency of iPSC generation, we found that ˜20% of EGFP (+) cells became TRA-1-60 (+) on day seven post-transduction (FIG. 2A). We confirmed that most of the iPSC colonies were derived from these TRA-1-60 (+) cells (FIG. 2B). We sorted the TRA-1-60 (+) cells on day 7, 11 and 15, and analyzed their gene expression by a microarray analysis. By comparing the gene expression between HDFs and human embryonic stem cells (ESCs), we selected 169 fibroblast-enriched genes (HDF-Gs), of which expression levels are higher at least 100-fold in HDF than in ESCs and 196 ESC-enriched genes (ES-Gs), of which expression levels are higher at least 100-fold in ESCs than in HDFs. We found that approximately half of these ES-Gs were increased at least 10-fold in the TRA-1-60 (+) cells compared to their levels in HDFs on day seven (FIG. 2C). These included well-known ESC marker genes, such as NANOG, and the endogenous OCT3/4, and their increased expression was confirmed by reverse transcription (RT)-PCR (FIG. 2D). In contrast, other ESC markers, such as LIN28 and endogenous SOX2, remained low. Approximately half of the HDF-Gs decreased by at least 10-fold (FIG. 2C). These data showed that TRA-1-60 (+) cells had acquired a partially reprogrammed state by day 7 post-transduction.

Unexpectedly, we also detected partial reprogramming in the EGFP (+) cells that stayed TRA-1-60 (−) (FIG. 2C). The DNA microarray analyses showed that, out of the 196 ES-Gs, the expression of 77 genes increased in EGFP (+)/TRA-1-60 (−) cells by at least 10-fold compared to their levels in HDFs on day seven. Among the 169 HDF-Gs, the expression levels of 65 genes decreased by at least 10-fold. In contrast, the expression of only a small numbers of ES-Gs and HDF-Gs changed >10-fold in the EGFP (−) cells (17 ES-Gs and 2 HDF-Gs). The changes in EGFP (+)/TRA-1-60 (−) cells were similar to, but slightly less prominent than, those in the TRA-1-60 (+) cells (FIG. 2E).

A principal component analysis (PCA) of ES-Gs and HDF-Gs also demonstrated partial reprogramming in TRA-1-60 (+) cells, as well as EGFP (+)/TRA-1-60 (−) cells, but not in EGFP (−) cells (FIG. 2F). Of note, we detected the progression of reprogramming in TRA-1-60 (+) cells on day 7, 11, and 15. In contrast, such progression was not seen in TRA-1-60 (−) cells. These data demonstrated that reprogramming was initiated in the majority of HDFs that had received high copy numbers of the OSKM transgenes, but that the maturation of reprogramming only took place in TRA-1-60(+) cells, not in EGFP (+)/TRA-1-60 (−) cells.

We then performed single cell RT-PCR with Taqman probes that quantitatively detected 13 ES-Gs and HDF-Gs (FIGS. 3A & B, Table 3).

TABLE 3
The number of analyzed cells by sigle cell PCR

| | | Analyzed |
|---|---|---|
| Cell Type | | cell number |
| HDF | | 121 |
| EGFP (−)/TRA-1-60 (−) | day 7 | 29 |
| | day 11 | 16 |
| | day 15 | 10 |
| EGFP (+)/TRA-1-60 (−) | day 7 | 33 |
| | day 11 | 26 |
| | day 15 | 15 |
| TRA-1-60 (+) | day 7 | 74 |
| | day 11 | 74 |
| | day 15 | 103 |
| | day 20 | 68 |
| | day 28 | 42 |

| | |
|---|---|
| ESC | 198 |

In the majority of TRA-1-60 (+) cells on day 7, the expression of 8 ES-Gs, including NANOG, L1TD1, GDF3, GAL, SALL4, APOE, CDH1 and EPCAM, increased at least 10-fold from the levels in HDFs. In contrast, the other five ES-Gs, including DPPA4, SOX2, LIN28, DNMT3B and GABRB3, remained suppressed until day 20 or 28. All four HDF-Gs (MMP1, DCN, LUM and CD13) were suppressed in the majority of TRA-1-60 (+) cells. The EGFP (+)/TRA-1-60 (−) cells showed similar, but smaller, changes. In sharp contrast, few changes were observed in the expression levels of ES-Gs and HDF-Gs in the EGFP (−) cells. A PCA demonstrated that the reprogramming in TRA-1-60 (+) cells gradually progressed from day 7 to day 28 (FIG. 3C). The TRA-1-60 (+) cells on day 7, 11, and 15 were more heterogenic in terms of their gene expression than original HDFs or ESCs (FIG. 3D). These data confirmed that reprogramming was initiated in most of the HDFs that had received high copy numbers of the OSKM transgenes, and that the reprogramming then progressed gradually and specifically in TRA-1-60 (+) cells.

In order to explore the fate of the nascent reprogrammed cells, we sorted TRA-1-60 (+) cells using magnetic activated cell sorting (MACS) on day 7, 11, 15 and 20, and re-plated them on SNL feeders. We counted the numbers of iPSC colonies 20 days after seeding (FIG. 4A). The efficiency of iPSC colony formation from TRA-1-60 (+) cells, which were sorted on day 7 or 11, remained low (˜1%). In contrast, the TRA-1-60 (+) cells sorted on day 15 or 20 showed a significantly increased efficiency of iPSC colony formation, indicative of the maturation of reprogramming from day 11 to 15.

To further trace the fate of TRA-1-60 (+) cells after sorting, we had to distinguish re-plated human cells from mouse feeder cells. To this end, we used a human specific antigen, TRA-1-85. When ESCs or established iPSCs were sorted for TRA-1-60 and were re-plated, more than 99% remained positive 4 days after re-seeding (FIG. 4B). In contrast, when TRA-1-60 (+) cells were sorted and re-plated on day 7 after transduction, ˜50% of them reverted and became TRA-1-60 (−) within 4 days after re-plating. The TRA-1-60 (+) cells sorted on day 11 also showed a strong tendency toward reversion. In contrast, the TRA-1-60 (+) cells sorted on day 15 showed less than 10% reversion (FIG. 4B). Thus, the degree of reversion and the efficiency of iPSC colony formation showed a reverse correlation.

The PCA of 196 ES-Gs and 169 HDF-Gs confirmed that there was a reversion in reprogramming in cells that reverted to TRA-1-60 (−) fate (FIG. 4C). Compared to TRA-1-60 (+) cells sorted on day 11, cells reverted to negative on day 15 and day 20 showed progressive changes in gene expression back to HDFs. In contrast, in the cells that remained TRA-1-60 (+) on days 15 and 20, we detected the progression of reprogramming in the gene expression pattern.

We then investigated the effects of reported pro-reprogramming factors on various aspects of iPSC generation, including the proliferation of fibroblasts, conversion to TRA-1-60 (+) cells, proliferation of TRA-1-60 (+) cells, death of TRA-1-60 (+) cells and reversion. We examined the effects of NANOG (Yu, J. et al. (2007) Science 318, 1917-20; Hanna, J. et al. (2009) Nature 462, 595-601; Silva, J. et al. (2006) Nature 441, 997-1001), LIN28 (Yu et al. (2007), supra), Cyclin D1 (Edel, M. J. et al. (2010) Genes Dev 24, 561-73), and p53 shRNA (Hanna et al. (2009), supra; Hong, H. et al. (2009) Nature 460, 1132-5; Kawamura, T. et al. (2009) Nature 460, 1140-4; Utikal, J. et al. (2009) Nature 460, 1145-8; Marion, R. M. et al. (2009) Nature 460, 1149-1153; Li, H. et al. (2009) Nature 460, 1136-9). We introduced each of these factors, together with OSKM, into HDFs and counted the numbers of iPSC colonies 28 days after transduction. We found that all of these factors, except for NANOG, significantly increased the number of iPSC colonies (FIG. 5A). The proliferation of HDFs was increased by Cyclin D1 and the p53 shRNA, but not by NANOG or LIN28 (FIG. 5B). The conversion to a TRA-1-60 (+) status was enhanced by LIN28, but not by NANOG, Cyclin D1 or the p53 shRNA (FIG. 5C). The proliferation of TRA-1-60 positive cells was also enhanced by LIN28 (FIG. 5D). Thus, the increased conversion may be attributable to the selective expansion of TRA-1-60 (+) cells. The death of TRA-1-60 (+) cells was suppressed by p53 shRNA (FIG. 5E). Reversion from TRA-1-60 (+) to (−) state was suppressed by LIN28 (FIG. 5F). These data demonstrated that each pro-reprogramming factor has a different mode of action during iPSC generation.

In the same manner as described above, Oct3/4, Sox2, Klf4 and c-Myc were introduced into human fibroblasts (TIG119 or TIG120) using retroviruses to reprogram the fibroblasts (d0).

On day 11 after gene introduction (d11), TRA-1-60 (+) cells were sorted out by the method mentioned above, and seeded onto mitomycin C-treated SNL cells. Ten (d21) or 18 (d29) days thereafter, TRA-1-60 (+) cells were sorted out in the same manner, and differentiated into nerve precursor cells by SFEBq method.

The differentiation resistance was evaluated by the number of the residual TRA-1-60 (+) cells. In addition, TRA-1-60 (+) cells were sorted out on 17 days after seeding (d28), reseeded onto mitomycin C-treated SNL cells, passaged 5 times (p5) or 10 times (p10) and evaluated for differentiation resistance in the same manner. Subculture was performed according to Takahashi et e al. (Cell (2006), supra).

After differentiation induction into nerve precursor cells by SFEBq method, the cells were dispersed into single cells and the number of TRA-1-60 (+) cells was measured using a flowcytometer. The content (%) of TRA-1-60 (+) cells was shown in Table 4. These data demonstrated that passage after sorting TRA-1-60 positive cells is important for producing iPS cells having low differentiation resistance.

TABLE 4

| | 1st | 2nd | 3rd | 4th | 5th |
|---|---|---|---|---|---|
| 201B7 | 0.19% | 0.47% | | | |
| TIG108-4f3 | 39.42% | 10.12% | | | |
| d 21 | 7.6% | 3.14% | 39.89% | 10.72% | |
| d 29 | 28.62% | 7.05% | 24.64% | 4.91% | 3.11% |
| p5 | 4.3% | 3.5% | 2.15% | 1.5% | |
| p10 | 2.81% | 1.68% | 1.14% | | |

201B7 is a standard strain that has been confirmed to be differentiation-sensitive (Takahashi et e al. Cell (2006), supra), whereas TIG108-4f3 is a standard strain that has been confirmed to be differentiation-resistant

### Discussion

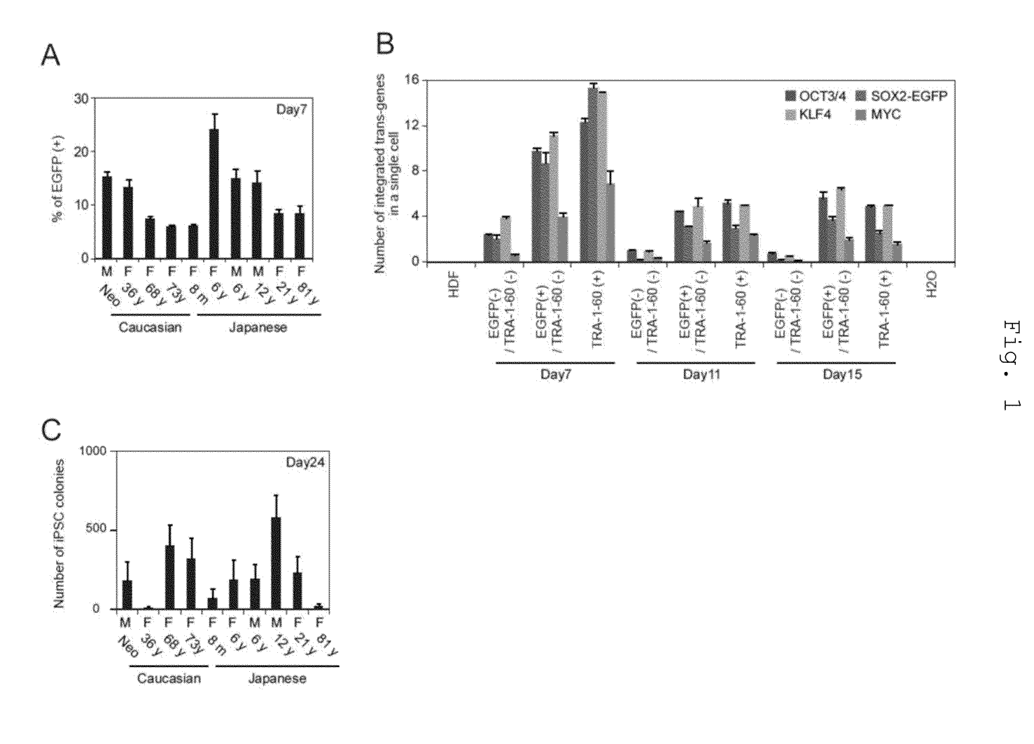

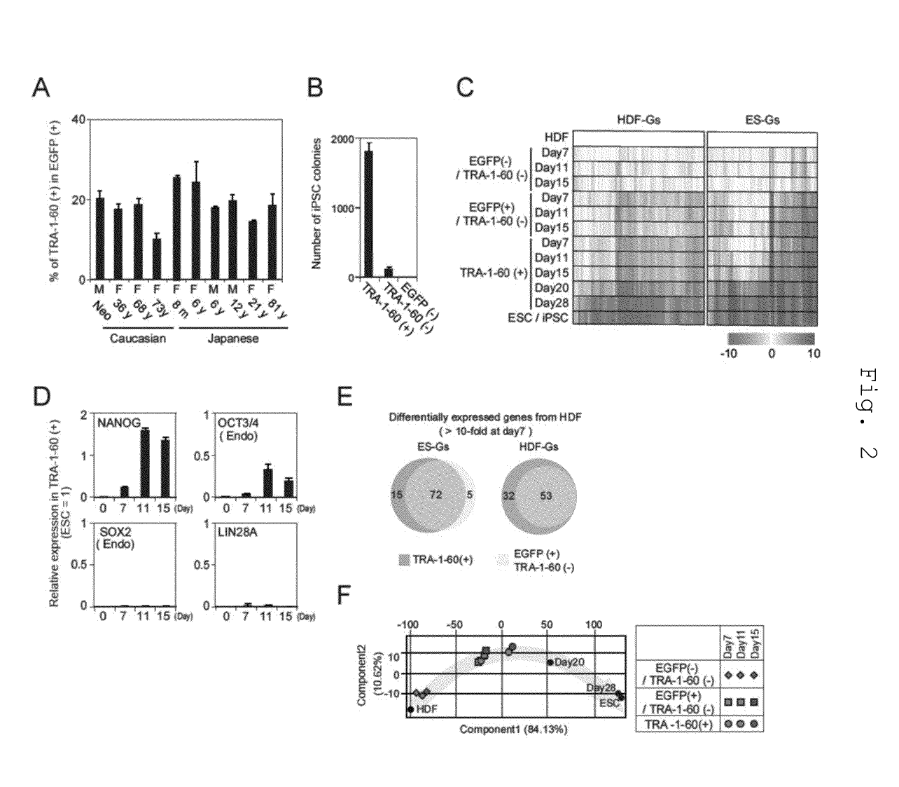

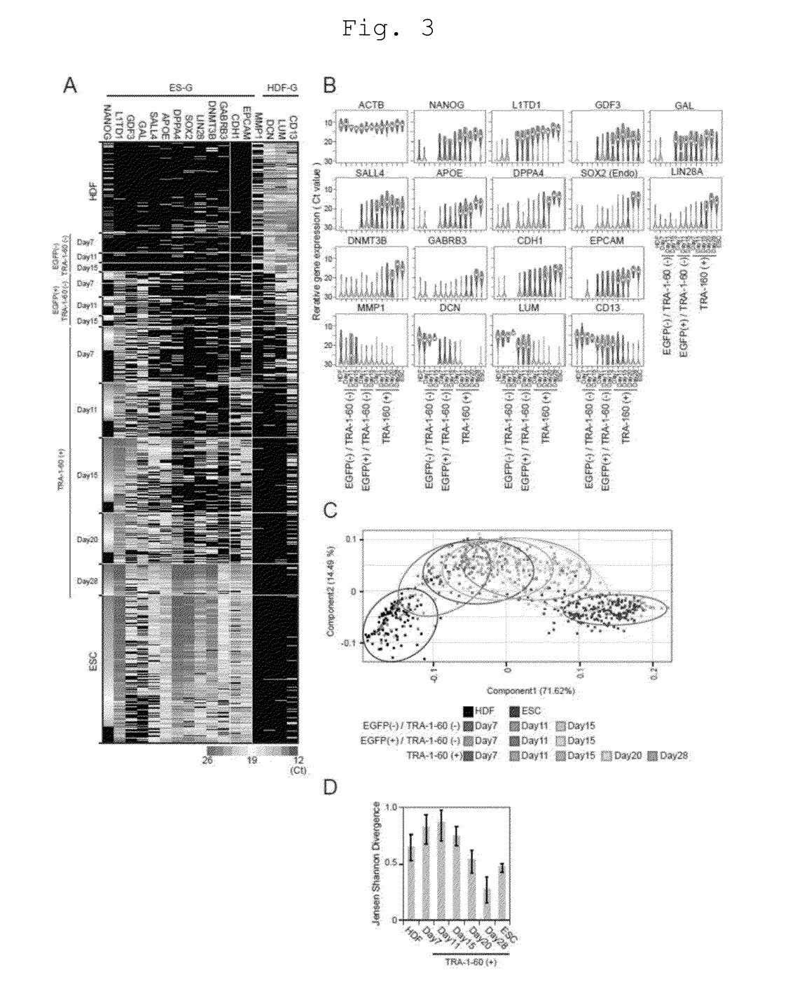

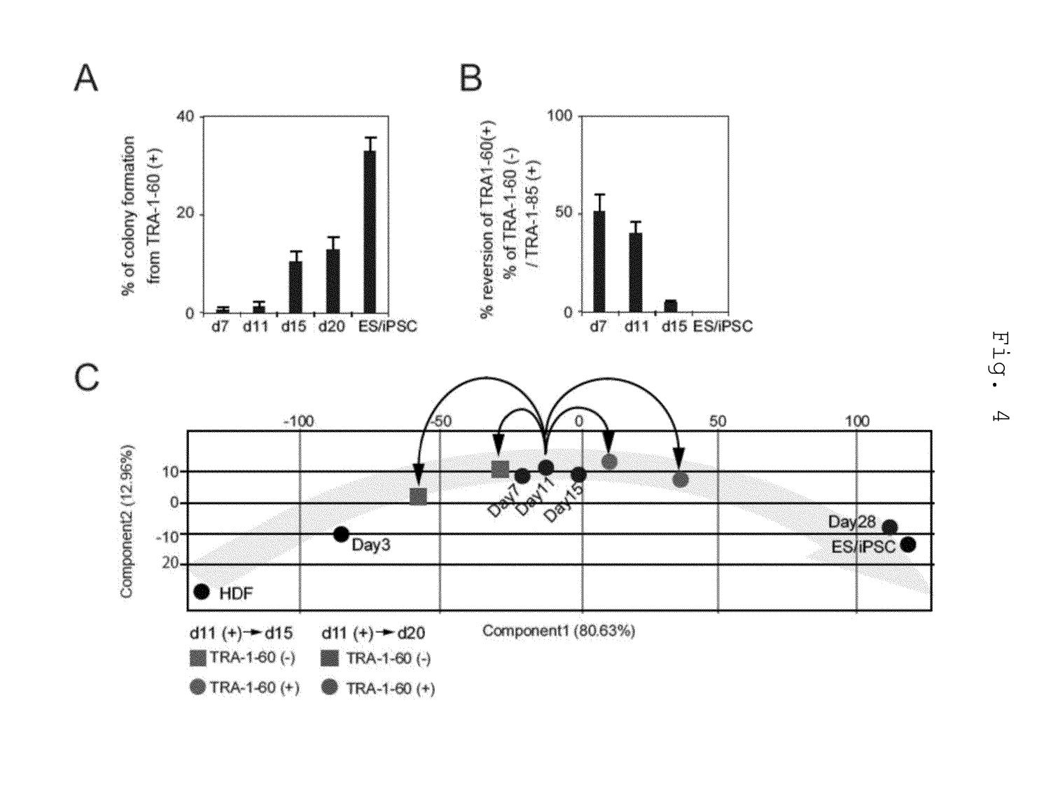

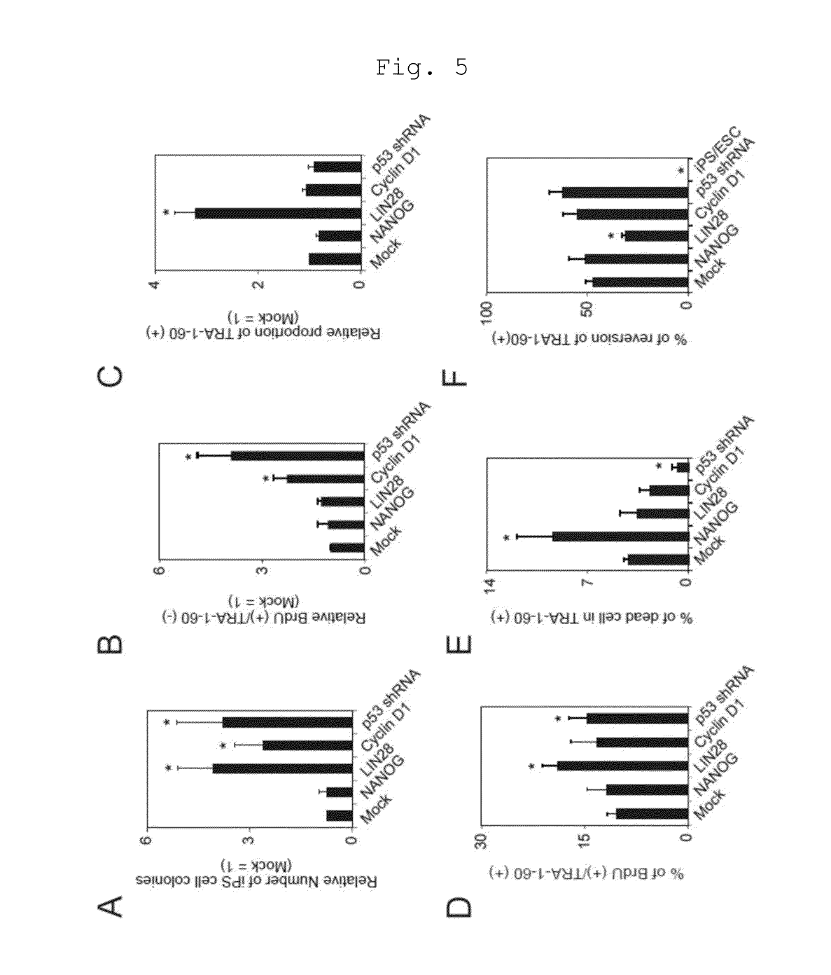

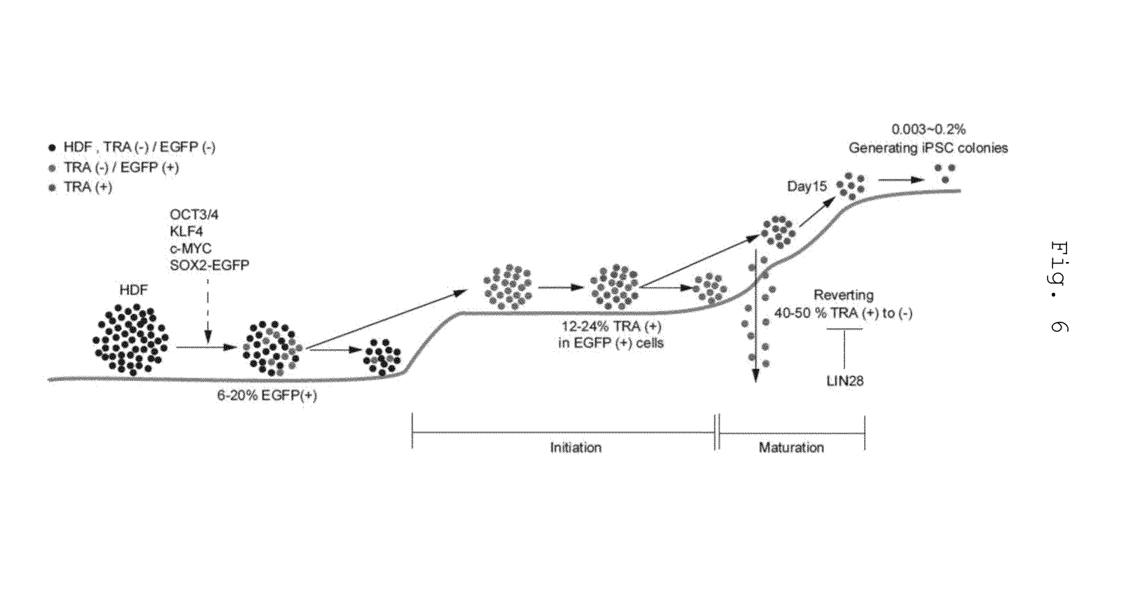

In the current study, we showed that reprogramming was initiated much more frequently than was previously anticipated in human fibroblasts that received the OSKM reprogramming factors (FIG. 6). We detected rapid induction of many ES-Gs and suppression of HDF-Gs in the majority of HDFs transduced with high copy numbers of OSKM retroviruses, indicating that reprogramming had been initiated. Approximately 20% of these transduced HDFs became positive for TRA-1-60, one of the best known markers of pluripotent stem cells, within seven days after transduction. These TRA-1-60 (+) cells showed progressive changes in their gene expression patterns toward those in iPSCs/ESCs. However, only a small portion of TRA-1-60 (+) cells completed the reprogramming process and became iPSCs. Thus, it is maturation, not initiation, that is responsible for the low efficiency of iPSC generation.

We also showed that one important mechanism underlying the inability of TRA-1-60 (+) cells to complete reprogramming is their reversion to a TRA-1-60 (−) state. When TRA-1-60 (+) cells were sorted and re-plated on SNL feeder cells on day seven, less than half of them remained positive four days after re-seeding. Since the proliferation of the reverted TRA-1-60 (−) cells was significantly lower than that of the positive cell (data not shown), the actual proportion of cells that reverted to a TRA-1-60 (−) state should be higher than 50%. When cells were sorted on day 11, the reversion rate was still high. In contrast, when they were sorted on day 15, the reversion rate became less than 10%. This result indicates that nascent reprogrammed cells mature during this period (between day 11 and 15).

Another important finding of this study is that each pro-reprogramming factor has a different mode of action in promoting iPSC generation. We found that three factors, LIN28, Cyclin D1 and p53 shRNA, significantly increased the numbers of iPSC colonies when co-transduced with OSKM. However, NANOG failed to show pro-reprogramming activity in our assay. Among the three factors that did show pro-reprogramming activities, Cyclin D1 and p53 shRNA increased the numbers of iPSC colonies mainly by promoting their proliferation and suppressing cell death. In contrast, LIN28 promoted the formation of TRA-1-60 (+) cells and inhibited their conversion back into (−) cells. We found that the endogenous LIN28 was activated later during reprogramming when the TRA-1-60 (+) cells were maturing. Thus LIN28 seems to promote the maturation of reprogramming. Of note, we found that LIN28 promotes the proliferation of TRA-1-60 (+) cells, but not TRA-1-60 (−) cells. This specific activation of nascent reprogrammed cells should contribute to the pro-reprogramming function of LIN28.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

The contents disclosed in any publication cited herein, including patents and patent applications, are hereby incorporated in their entireties by reference, to the extent that they have been disclosed herein.

1. A method of producing induced pluripotent stem (iPS) cells, which comprises the following steps:
(i) a step for introducing reprogramming factors into somatic cells;
(ii) a step for culturing the cells obtained in step (i) for more than 11 days and not more than 29 days;
(iii) a step for sorting TRA-1-60-positive cells from the cells obtained in step (ii);
(iv) a step for culturing the TRA-1-60-positive cells sorted in step (iii);
(v) a step for transferring a colony obtained in step (iv) to another culture vessel; and
(vi) a step for culturing the cells obtained in step (v), thereby obtaining iPS cells.

2. The method according to claim 1, wherein the reprogramming factors comprise:
(a) Oct3/4 or a nucleic acid encoding same;
(b) Sox2 or a nucleic acid encoding same; and
(c) Klf4 or a nucleic acid encoding same.

3. The method according to claim 2, wherein the reprogramming factors further comprise (d) Lin28 or a nucleic acid encoding same.

4. The method according to claim 1, wherein the iPS cells are human iPS cells.

5. The method according to claim 1, wherein the culture period of step (ii) is 15 to 20 days.

6. The method according to claim 1, wherein the cells obtained in step (vi) are subcultured 10 times or more.

7. A method of producing a population of differentiated cells that has a reduced rate of residual undifferentiated cells, which comprises inducing differentiation of iPS cells obtained by the method according to claim 6.
